(19) United States
(12) Patent Application Publication
(10) Pub. No.: US 2020/0100933 A1
(43) Pub. Date: Apr. 2, 2020

(54) METHODS AND SYSTEMS FOR CONCUSSION MANAGEMENT USING COLD STIMULUS

(71) Applicant: Cornell University, Ithaca, NY (US)

(72) Inventor: Barry Kosofsky, New York, NY (US)

(73) Assignee: Cornell University, Ithaca, NY (US)

(21) Appl. No.: 16/702,232

(22) Filed: Dec. 3, 2019

Related U.S. Application Data
(63) Continuation of application No. PCT/US2019/017517, filed on Feb. 11, 2019.
(60) Provisional application No. 62/629,373, filed on Feb. 12, 2018.

(51) Int. Cl. A61F 7/00

(52) U.S. Cl. CPC A61F 7/00; A61F 2007/0036; A61F 2007/0056

(57) ABSTRACT

The present disclosure describes systems and methods to non-invasively determine whether a patient has an autonomic dysfunction. In some implementations, the autonomic dysfunction can be a neurological dysfunction. The system can measure a patient's autonomic nervous system response to a stimulus. The stimulus can be a thermal stimulus, such as a cold temperature. The system can apply the thermal stimulus to an extremity of the patient, such as the patient's hand or arm.

### CROSS-REFERENCE TO RELATED APPLICATIONS

This application is a continuation of PCT/US2019/017517, filed on Feb. 11, 2019, which claims priority to U.S. Provisional Patent Application No. 62/629,373, filed on Feb. 12, 2018, both of which are herein incorporated by reference in their entireties.

### BACKGROUND OF THE DISCLOSURE

A concussion or mild traumatic brain injury (i.e., mTBI) is a complex pathophysiologic process that is caused by traumatic biomechanical forces to the head. Due to their complexity, concussions are difficult to diagnose.

### SUMMARY OF THE DISCLOSURE

According to at least one aspect of the disclosure, a system can include a thermal unit to maintain a predetermined temperature and expose a subject to a thermal stimulus. The system can include an interface to receive one or more input signals indicating one or more vital signs of the subject in response to exposure to the thermal stimulus. The vital signs can include at least one of a blood pressure of the subject or a heart rate of the subject. The system can include a condition detection engine to determine a baseline of the one or more vital signs over a first portion of the one or more input signals. The condition detection engine can detect, at a second portion of the one or more input signals, a change from the baseline of the one or more vital signs over the first portion of the one or more input signals. The condition detection engine can generate an alarm condition indicating an autonomic dysfunction based on the change from the baseline of the one or more vital signs over the first portion of the one or more input signals.

In some implementations, the autonomic dysfunction can be a concussion. The baseline of the one or more vital signs can include a first baseline of the heart rate of the subject and a second baseline of the blood pressure of the subject.

The condition detection engine can determine a first baseline of the heart rate over the first portion of the one or more input signals. The condition detection engine can determine a second baseline of the blood pressure over the first portion of the one or more input signals. The condition detection engine can generate the alarm condition indicating the autonomic dysfunction based on a change of the first baseline during the second portion of the one or more input signals and a change of the second baseline during the second portion of the one or more input signals.

The condition detection engine can determine a baseline of the one or more vital signs over a portion of one or more control input signals received that are generated prior to the interface to receive one or more input signals indicating one or more vital signs of the subject in response to exposure to the thermal stimulus. The condition detection engine can generate the alarm condition indicating the autonomic dysfunction based on a change from the baseline of the one or more vital signs over a portion of one or more control input signals generated prior to the interface receiving the one or more input signals indicating the one or more vital signs of the subject in response to exposure to the thermal stimulus.

The condition detection engine can determine a derived vital sign based on the one or more vital signs of the subject. The derived vital sign comprises an R to R heart rate interval, a heart rate variability, or a root mean squared value of the heart rate. The thermal stimulus can be between −10° C. and 10° C. The thermal unit can include a thermoelectric thermal stimulator to maintain the thermal stimulus. The thermal unit can include a thermal stimulator conformed to receive a hand of the subject and expose the hand of the subject to the thermal stimulus.

According to at least one aspect of the disclosure, a method can include receiving a first portion of one or more input signals indicating one or more vital signs of a subject. The vital signs can include at least one of a blood pressure of the subject or a heart rate of the subject. The method can include exposing the subject to a thermal stimulus. The method can include receiving a second portion of the one or more input signals indicating the one or more vital signs of the subject. The second portion of the one or more input signals can include a response to the thermal stimulus. The method can include determining a baseline of the first portion of one or more input signals indicating the one or more vital signs of the subject. The method can include determining a change from the baseline of the one or more vital signs over the first portion of the one or more input signals in the second portion of the one or more input signals. The method can include generating an alarm condition indicating an autonomic dysfunction based on the change from the baseline of the one or more vital signs over the first portion of the one or more input signals in the second portion of the one or more input signals.

In some implementations, the autonomic dysfunction is a concussion. The baseline of the one or more vital signs can include a first baseline of the heart rate of the subject and a second baseline of the blood pressure of the subject. In some implementations, the method can include determining a first baseline of the heart rate over the first portion of the one or more input signals. The method can include determining a second baseline of the blood pressure over the first portion of the one or more input signals. The method can include generating the alarm condition indicating the autonomic dysfunction based on a change of the first baseline during the second portion of the one or more input signals and a change of the second baseline during the second portion of the one or more input signals.

In some implementations, the method can include determining a baseline of the one or more vital signs over a portion of one or more control input signals received that are generated prior to the interface receiving the one or more input signals indicating one or more vital signs of the subject in response to exposure to the thermal stimulus. The method can include generating the alarm condition indicating the autonomic dysfunction based on a change from the baseline of the one or more vital signs over a portion of one or more control input signals received that are generated prior to the interface to receive one or more input signals indicating one or more vital signs of the subject in response to exposure to the thermal stimulus.

The method can include determining a derived vital sign based on the one or more vital signs of the subject. The derived vital sign can include an R to R heart rate interval, a heart rate variability, or a root mean squared value of the heart rate. The thermal stimulus can be between −10° C. and 10° C. The method can include exposing the subject's hand to the thermal stimulus. The method can include exposing the subject to the thermal stimulus for between 1 minute and 5 minutes.

The foregoing general description and following description of the drawings and detailed description are exemplary and explanatory and are intended to provide further explanation of the invention as claimed. Other objects, advantages, and novel features will be readily apparent to those skilled in the art from the following brief description of the drawings and detailed description.

### BRIEF DESCRIPTION OF THE DRAWINGS

The accompanying drawings are not intended to be drawn to scale. Like reference numbers and designations in the various drawings indicate like elements. For purposes of clarity, not every component may be labeled in every drawing. In the drawings:

FIG. 1 illustrates an example system to detect neurologic dysfunction.

FIGS. 2A-2D illustrate example thermal units that can be used in the example system illustrated in FIG. 1.

FIG. 3 illustrates a block diagram of an example method to detect neurological dysfunction using the example system illustrated in FIG. 1.

FIG. 4 illustrates example data collected with a system similar to the example system illustrated in FIG. 1.

### DETAILED DESCRIPTION

The various concepts introduced above and discussed in greater detail below may be implemented in any of numerous ways, as the described concepts are not limited to any particular manner of implementation. Examples of specific implementations and applications are provided primarily for illustrative purposes.

The present disclosure describes systems and methods to non-invasively determine whether a patient has an autonomic dysfunction. In some implementations, the autonomic dysfunction can be a neurological dysfunction. The system can measure a patient's autonomic nervous system response to a stimulus. The stimulus can be a thermal stimulus, such as a cold temperature (e.g., 0° C.) or a hot temperature (e.g., 100° C.). The system can apply the thermal stimulus to an extremity of the patient, such as the patient's hand or arm.

The system can measure the patient's autonomic nervous system response to the thermal stimulus to diagnose the patient with an autonomic dysfunction. In some implementations, the autonomic dysfunction can be a concussion. The system can measure one or more vital signs as the system exposes the patient to the thermal stimulus. The vital signs can include blood pressure or heart rate. The system can identify a probability of the patient having an autonomic dysfunction based on the patient's autonomic response to the thermal stimulus.

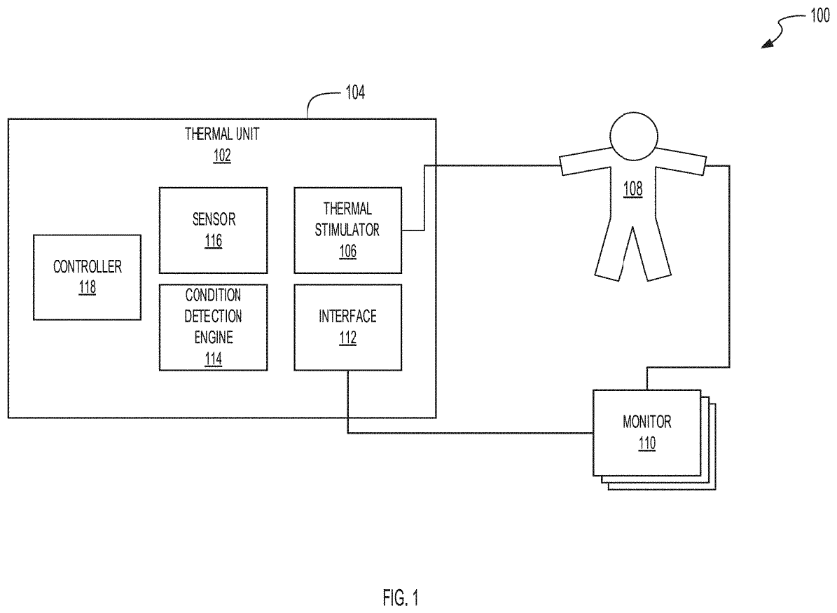

FIG. 1 illustrates an example system 100 to detect neurological dysfunctions such as an autonomic dysfunction. The system 100 can be used to detect dysfunction in the sympathetic or parasympathetic nervous system. The system 100 can include a thermal unit 102 that can expose a subject 108 to thermal stimulus. For example, the thermal unit 102 can expose the subject's hand to cold temperatures, such as 0° C. The thermal unit 102 can include a thermal stimulator 106 that can impart the predetermined thermal stimulus on the subject 108. The thermal unit 102 can include a controller 118 that can measure the temperature of the thermal stimulator 106 via one or more sensors 116 and control the temperature of the thermal stimulator 106. The system 100 can include one or more monitors 110 that can measure vital signs of the subject 108. The monitors 110 can transmit the signal that includes the measured vital signs to the thermal unit 102, which can receive the signals via one or more interfaces 112.

The system 100 can include one or more thermal units 102. The thermal unit 102 can be a bench top device, portable device, or wearable device that provides a thermal stimulus to the subject 108 and measures or records the subject's response to the thermal stimulus. The thermal unit 102 can, based on the subject's response to the stimulus, make a prediction whether the user has an autonomic dysfunction. In some implementations, the thermal unit 102 can expose the subject 108 to a thermal stimulus, such as a cold temperature (e.g., a temperature between −10° C. and 10° C.). In some implementations, the thermal unit 102 can expose the subject 108 to a thermal stimulus that can include hot or elevated temperatures. For example, the elevated temperatures can be elevated with respect to the subject's body temperature (e.g., the thermal stimulation can be a temperature above about 100° C.).

The thermal unit 102 can include one or more thermal stimulators 106. The thermal stimulators 106 can be within an enclosure 104 of the thermal unit 102. For example, within the enclosure 104, the thermal unit 102 can include a volume that can be configured to receive a portion or extremity of the subject's body (e.g., the subject's hand). The thermal stimulator 106 can generate the thermal stimulus that the thermal unit 102 exposes to the subject 108. In some implementations, the thermal stimulator 106 can be within the volume that receives the subject's, for example, hand and can be configured to come into contact with the subject's extremity. The thermal stimulator 106 can include a gel membrane covering at least a portion of the outer surface of the thermal stimulator 106. The gel membrane can include an anti-freeze gel. The gel membrane can be configured to improve thermal transmission between the thermal stimulator 106 and the subject's extremity. For example, the gel membrane can be pliable to form around a portion of the subject's extremity and enable a greater surface area of the subject's extremity to come into contact with the cooling surface of the thermal stimulator 106. In some implementations, rather than directly exposing the subject 108 to the thermal stimulus, the thermal stimulator 106 can cool a medium that is exposed to the subject's extremity. For example, the thermal stimulator 106 can cool (or heat) a liquid or fluid that is pumped to the volume that receives the subject's body.

The thermal stimulator 106 can include a thermoelectric cooler. The thermoelectric cooler can include or can be a solid-state refrigerator, such as a heat pump or Peltier device. For example, the thermal stimulator 106 can include a Peltier device and a voltage can be applied to the Peltier device. The activated Peltier device can pump heat from one side of the device to the other side. For example, the Peltier device can pump heat from the volume that receives the subject's body to outside of the volume, which can be referred to as the “hot side” of the Peltier device. The hot side of the Peltier device can be coupled with a heat sink that can dissipate the heat transferred to the hot side. The thermoelectric cooler can chill and circulate a liquid to the thermal stimulator 106. The liquid can be an antifreeze, non-freezing gel, water, or other fluid.

In some implementations, the thermal stimulator 106 can include a removable thermal mass. The thermal mass can include a gel or liquid that can be pre-frozen, cooled, or heated before use of the thermal unit 102. For example, the thermal mass can be removed from the thermal unit 102, frozen, and placed in the thermal unit 102 during the autonomic dysfunction test. In some implementations, the thermal stimulator 106 can generate temperatures between about −55° C. and about 105° C., between about −25° C. and about 45° C., between about −25° C. and about 25° C., or between about −10° C. and about 10° C. In some implementations, the thermal stimulator 106 can generate temperatures less than 10° C., 9° C., 8° C., 7° C., 6° C., 5° C., 4° C., 3° C., 2° C., 1° C., 0° C., −1° C., −2° C., −3° C., −4° C., −5° C., −6° C., −7° C., −8° C., −9° C. or −10° C.

The thermal unit 102 can include one or more controllers 118. The controllers 118 can be or can include processors. The processor 200 can include a microprocessor, ASIC, FPGA, combinations thereof, or other control logic. The processor can be a multi-core processor or an array of processors. The controller 118 can control the thermal stimulator 106. For example, the controller 118 can receive an input temperature from a user via a user interface of the thermal unit 102. The controller 118 can activate the thermal stimulator 106 by supplying voltage or current to the, for example, Peltier device of the thermal stimulator 106. In some implementations, the controller 118 can set the thermal stimulator 106 to a temperature specified by a health care professional. For example, the health care professional can program the temperature via a graphical interface. In some implementations, the controller 118 can set the thermal stimulator 106 to a temperature based on a response from the subject 108. The response can be a physiological response. For example, if the target temperature (as set by a health care professional) is 0° C., the controller 118 can initially set the temperature of the thermal stimulator 106 to room temperature and can ramp the temperature down to the target temperature over a predetermined time (e.g., 30 seconds, 1 minute, 2 minutes, or 5 minutes). The controller 118 can stop the transition towards the target temperature and maintain the temperature at the stopped temperature when the condition detection engine 114 detects a physiological response from the subject (e.g., a change in heart rate) or when the subject activates, for example, a button. The subject can activate the button to indicate that the thermal stimulus has reached a pain threshold of the subject. In some implementations, the controller 118 can reduce the thermal stimulation to a predetermined level below the subject's pain threshold. For example, if the subject activates the button while the thermal stimulation is at 5° C. (when ramping down to 0° C.), the controller 118 can increase the thermal stimulation to 10° C. The controller 118 can set the thermal stimulation to a temperature between about 1% and about 20%, between about 1% and about 15%, or between about 5% and about 15% below the subject's pain threshold.

The thermal unit 102 can include one or more sensors 116. The sensors 116 can be temperature sensors. The sensors 116 can measure the temperature at the thermal stimulator 106. The controller 118 can receive a signal from the temperature sensor at the thermal stimulator 106 that indicates the temperature the thermal unit 102 is exposing to the subject 108. While the temperature measured by the sensor 116 at the thermal stimulator 106 is above the predetermined temperature of the thermal stimulus set by the health care professional (when a cold target temperature is set), the controller 118 can activate the thermal stimulator 106 to reduce the temperature of the thermal stimulator 106 until the thermal stimulator 106 reaches the target temperature of the thermal stimulator. Once the controller 118 determines the thermal stimulator 106 is at the target temperature, as measured by one of the sensors 116, the controller 118 can intermittently activate the thermal stimulator 106 to maintain the temperature at target thermal stimulation temperature. The thermal unit 102 can include one or more sensors 116 on the hot side (e.g., at a heat sink) of the thermal stimulator 106. As the volume that receives the subject's hand is cooled, the hot side of the thermal stimulator 106 can increase in temperature. The controller 118 can measure the temperature of the hot side to ensure that the hot side does not over heat. For example, if the controller 118 determines the hot side temperature is above a predetermined threshold, the controller 118 can de-activate the thermal stimulator 106.

The thermal unit 102 can include one or more interfaces 112. The interfaces 112 can be hardware or software interfaces to receive data from the monitors 110. The interfaces 112 can be hardware interfaces that can receive a wired connection from the monitors 110 over which data can be transmitted from the monitors 110 to the thermal unit 102. For example, the interface 112 can be a USB, network, serial, proprietary, or other type of hardware interface. The interfaces 112 can be or can include software interfaces. For example, the interfaces 112 can be wireless interfaces that enable the monitors 110 to transmit data to the thermal unit 102 wirelessly. The interface 112 can be a Bluetooth interface, Wi-Fi interface, NFC, or other wireless interface.

The system 100 can include one or more monitors 110. The monitors 110 can be components of or integrated into the thermal unit 102. The monitors 110 can be components separate from the thermal unit 102. For example, the monitors 110 can be separate devices that output data signals that are transmitted to and received by the thermal unit 102. The monitors 110 can include blood pressure monitors, heart rate, electrocardiography monitors, temperature monitors, blood flow monitors, pulse oximetry monitors, pulse monitors, or other type of monitor to measure biometric parameters of the subject 108. In some implementations, the monitor 110 can be a Finpres Nova® (made available by Finpres Medical Systems, the Netherlands). In some implementations, the monitor 110 can be a Caretaker System® (made available by CareTaker Medical, Charlottesville, Va.).

The thermal unit 102 can include one or more condition detection engines 114. The condition detection engine 114 can be an application, applet, script, service, daemon, routine, or other executable logic to determine a probability that the subject 108 has an autonomic dysfunction based on the one or more signals received from the monitors 110. The condition detection engine 114 can diagnose a patient with concussion, Neurocardiogenic Syncope (NCS), Multiple System Atrophy (MSA), Hereditary Sensory and Autonomic Neuropathies (HSAN), Holmes-Adie Syndrome (HAS), Postural Orthostatic Tachycardia Syndrome (POTS), Parkinson's disease, or other autonomic dysfunction. The condition detection engine 114 can determine a baseline of the one or more vital signs in the input signals received by the thermal unit 102 from the monitors 110. The condition detection engine 114 can determine the baseline over a first portion of the input signals. For example, the first portion of the input signals can be a portion of the input signals prior to the activation of the thermal stimulator 106 or before the subject 108 is exposed to the predetermined temperature stimulus. For example, the thermal unit 102 can receive input signals from the monitors 110 for between about 10 seconds and about 5 minutes, between about 30 seconds and about 5 minutes, between about 1 minute and about 5 minutes, between about 2 minutes and about 5 minutes, or between about 3 minutes and about 5 minutes prior to the activation of the thermal stimulator 106. In some embodiments, the thermal unit 102 can receive input signals from the monitors 110 for at least 15 seconds, 16 seconds, 17 seconds, 18 seconds, 19 seconds, 20 seconds, 21 seconds, 22 seconds, 23 seconds, 24 seconds, 25 seconds, 26 seconds, 27 seconds, 28 seconds, 29 seconds, 30 seconds, 31 seconds, 32 seconds, 33 seconds, 34 seconds, 35 seconds, 36 seconds, 37 seconds, 38 seconds, 39 seconds, 40 seconds, 41 seconds, 42 seconds, 43 seconds, 44 seconds, 45 seconds, 46 seconds, 47 seconds, 48 seconds, 49 seconds, 50 seconds, 51 seconds, 52 seconds, 53 seconds, 54 seconds, 55 seconds, 56 seconds, 57 seconds, 58 seconds, 59 seconds, 60 seconds, 61 seconds, 62 seconds, 63 seconds, 64 seconds, 65 seconds, 66 seconds, 67 seconds, 68 seconds, 69 seconds, 70 seconds, 71 seconds, 72 seconds, 73 seconds, 74 seconds, 75 seconds, 76 seconds, 77 seconds, 78 seconds, 79 seconds, 80 seconds, 81 seconds, 82 seconds, 83 seconds, 84 seconds, 85 seconds, 86 seconds, 87 seconds, 88 seconds, 89 seconds, 90 seconds, 91 seconds, 92 seconds, 93 seconds, 94 seconds, 95 seconds, 96 seconds, 97 seconds, 98 seconds, 99 seconds, 100 seconds, 101 seconds, 102 seconds, 103 seconds, 104 seconds, 105 seconds, 106 seconds, 107 seconds, 108 seconds, 109 seconds, 110 seconds, 111 seconds, 112 seconds, 113 seconds, 114 seconds, 115 seconds, 116 seconds, 117 seconds, 118 seconds, 119 seconds, 120 seconds, 121 seconds, 122 seconds, 123 seconds, 124 seconds, 125 seconds, 126 seconds, 127 seconds, 128 seconds, 129 seconds, 130 seconds, 131 seconds, 132 seconds, 133 seconds, 134 seconds, 135 seconds, 136 seconds, 137 seconds, 138 seconds, 139 seconds, 140 seconds, 141 seconds, 142 seconds, 143 seconds, 144 seconds, 145 seconds, 146 seconds, 147 seconds, 148 seconds, 149 seconds, 150 seconds, 151 seconds, 152 seconds, 153 seconds, 154 seconds, 155 seconds, 156 seconds, 157 seconds, 158 seconds, 159 seconds, 160 seconds, 161 seconds, 162 seconds, 163 seconds, 164 seconds, 165 seconds, 166 seconds, 167 seconds, 168 seconds, 169 seconds, 170 seconds, 171 seconds, 172 seconds, 173 seconds, 174 seconds, 175 seconds, 176 seconds, 177 seconds, 178 seconds, 179 seconds, 180 seconds, 181 seconds, 182 seconds, 183 seconds, 184 seconds, 185 seconds, 186 seconds, 187 seconds, 188 seconds, 189 seconds, 190 seconds, 191 seconds, 192 seconds, 193 seconds, 194 seconds, 195 seconds, 196 seconds, 197 seconds, 198 seconds, 199 seconds, 200 seconds, 205 seconds, 210 seconds, 215 seconds, 220 seconds, 230 seconds, 240 seconds, 250 seconds, 260 seconds, 270 seconds, 280 seconds, 290 seconds or 300 seconds, or more than 300 seconds.

The condition detection engine 114 can detect a second portion of the input signals from the monitors 110. The second portion of the input signals can include a change from the baseline. The second portion of the input signals can include values indicating vital signs recorded during the subject's exposure to the thermal stimulus by the thermal stimulator 106. In some implementations, the condition detection engine 114 can detect the change from the baseline determined for the first portion of the input signals. For example, the condition detection engine 114 can determine, for example, an average heart rate during the first phase of the input signals. The condition detection engine 114 can determine the change in baseline as an increase from the average of the first portion of the input signals. In some implementations, the condition detection engine 114 can detect a change from the baseline when the values in the input signal cross a predetermined threshold (e.g., between about two and six standard deviations above the values in the first portion of the input signal).

In some implementations, the condition detection engine 114 can identify the first and second portions of the input signals based on timestamps associated with the input signals. For example, the controller 118 can save to a memory of the thermal unit 102 a timestamp that indicates that the controller 118 activated the thermal stimulator 106 60 seconds into the test session (e.g., the first portion of the input signals has a duration of 60 seconds). A user can program or otherwise set the duration of the time after the thermal unit 102 begins receiving data from the monitors 110 that the thermal unit 102 activates the thermal stimulator 106, which is set to 60 seconds in the above example. The monitors 110 can sample the vital signals at a predetermined sampling frequency (e.g., 256 Hz) to generate input signals with samples occurring at the predetermined sampling frequency. The condition detection engine 114 can multiply the sampling frequency by the duration of the first portion of the input signals to determine a count of the samples in the first portion of the input signals (e.g., in this example 256×60=15,360) and at what sample in the input signals the second portion of the input signals begins (e.g., 15,361). The condition detection engine 114 can read the samples of the input samples prior to the calculated count of samples as part of the first portion of the input signals and the samples after the calculated count of samples as part of the second portion of input signals. In some implementations, the condition detection engine 114 can divide the input signals into first and second input signals that the condition detection engine 114 can save to memory as separate files.

In some implementations, the controller 118 can generate an output signal that includes values that indicate the state of the thermal stimulator 106. The output signal can have the same sampling frequency as the sampling frequency of the monitors 110. For example, the controller 118 can output an output signal that includes value of 0 when the thermal stimulator 106 is not activated and value of 1 when the thermal stimulator is active. Because the output signal has the same sampling frequency as the input signals from the monitors 110, the output signal from the controller 118 indicating the state of the thermal stimulator 106 can be time-locked with the input signals from the monitors 110. The condition detection engine 114 can read the output signal from the controller 118 to determine whether the samples in the input signals correspond to times when the thermal stimulator was not active (e.g., the above-referenced first portion of the input signals) or when the thermal stimulator 106 was active (or after the activation of the thermal stimulator 106), which can be the above-referenced second portion of the input signals.

In some implementations, the condition detection engine 114 can include one or more machine learning models to classify the input signals into a diagnostic or condition category. Each of the condition categories can correspond to a different autonomic dysfunction. The input signals can be input features for the machine learning models. In some implementations, the machine learning model can be a neural network.

The condition detection engine 114 can generate alarm conditions. The alarm condition can indicate that the subject 108 has an autonomic dysfunction. The alarm condition can indicate a probability that the subject 108 has an autonomic dysfunction. The condition detection engine 114 can determine the subject 108 has an autonomic dysfunction and generate an alarm condition based on a change from the baseline (or lack thereof) during the second portion of the input signals. The condition detection engine 114 can determine whether an autonomic dysfunction exists based on an amount of change between the first portion of the input signals and the second portion of the input signals.

For example, the condition detection engine 114 can determine whether an autonomic dysfunction exists based on the amplitude of the change in the values between the first portion of the input signals and the second portion of the input signals. For example, the condition detection engine 114 can determine an average of the sample values in the first portion of the input signals (e.g., a baseline value). The condition detection engine 114 can determine test values that can include an average of the sample values in the second portion of the input signals, a maximum of the sample values in the second portion of the input signals, an average of the sample values in the second portion of the input signals and during the activation of the thermal stimulator 106, a maximum of the sample values in the second portion of the input signals and during the activation of the thermal stimulator 106, an average of the sample values in the second portion of the input signals and after the activation of the thermal stimulator 106, or a maximum of the sample values in the second portion of the input signals and after the activation of the thermal stimulator 106. The condition detection engine 114 can be based on the magnitude of the change between the baseline values and the test values. The condition detection engine 114 can determine there is an impairment to the nervous system based on a blunted or reduced response to the thermal stimulation. For example, the condition detection engine 114 determines the magnitude of the change between the baselines values and the test values does not cross a predetermined threshold. The predetermined threshold can be, for example, between about two and about six standard deviations above the values of the samples in the first portion of the input signals. In some implementations, the condition detection engine 114 can determine the change as the difference between the baseline and the sample values at a predetermined length of time after the activation of the thermal stimulator 106. For example, the condition detection engine 114 can determine the change as the difference between the average of the baseline values and the average of between about 1 second and about 2 minutes, between about 1 second and about 1.5 minutes, between about 1 second and about 1 minute, between about 5 seconds and about 1 minute, between about 10 seconds and about 1 minute, between about 15 seconds and about 1 minute, or between about 30 seconds and about 1 minute of data. The predetermined length of time after the activation of the thermal stimulator 106 for measuring the difference between about 30 seconds, 1 minute, 2 minutes, 3 minutes, 4 minutes, 5 minutes, 10 minutes, 15 minutes, 20 minutes, or about 30 minutes after the activation of the thermal stimulator 106. For example, the condition detection engine 114 can determine that the subject 108 has an autonomic dysfunction if the average value of the subject's heart rate during a 30-second timeframe 5 minutes after the thermal stimulator 106 is deactivated is not within a predetermined range (e.g., between about one and about six standard deviations) of the baseline value that the condition detection engine 114 calculated for the first portion of the input signals.

In some implementations, the condition detection engine 114 can determine whether an autonomic dysfunction exists based on a response time or delay of a change in the sample values between the first and second portions of the input signals. For example, the condition detection engine 114 can determine the response time or delay as a time between the activation of the thermal stimulator 106 and when an average of the samples values in the second portion of the input signals crosses a predetermined threshold, reaches a maximum value, or returns to a baseline value. The average can be the average of a moving window of data within the second portion of the input signals. For example, the moving window average can be the average of, for example, between about 1 second and about 2 minutes, between about 1 second and about 1.5 minutes, between about 1 second and about 1 minute, between about 1 second and about 30 seconds, between about 1 second and about 20 seconds, between about 1 second and about 15 seconds, between about 1 second and about 10 seconds, between about 1 second and about 5 seconds, or between about 1 second and about 2 seconds of data in the second portion of the input signals. The condition detection engine 114 can calculate the moving window average for the samples and shift the moving window between about 50% and about 100%. The predetermined threshold can be a percentage or standard deviation increase over the values of the samples in the first portion of the input string. For example, the condition detection engine 114 can determine a time that the moving window average of the heart rate values in the second portion of the input signals increases by one standard deviation above the sample values in the first portion of the input signal that includes the heart rate values.

The condition detection engine 114 can determine the delay as the amount of time until the values in the second portion of the input signals returns to a baseline value, which can be referred to as a reset time. For example, the condition detection engine 114 can determine the baseline value as an average of the sample values in the first portion of the input signals. The condition detection engine 114 can calculate the amount of time until the input signals return to the baseline as the length of time between the activation of the thermal stimulator 106 and when the sample values in the second portion of the input signals return to within a predetermined range of the baseline values. For example, the condition detection engine 114 can set a threshold at two standard deviations above the baseline values. The condition detection engine 114 can determine a time (e.g., by counting the number of samples in the second portion of the input signals after the activation of the thermal stimulator 106) when the values in the second portion of the input signal cross below the threshold of two standard deviations. The condition detection engine 114 can determine the delay as the amount of time between the activation of the thermal stimulator 106 and when the sample values within the second portion of the input signals return to within one, two, three, five, or six standard deviations of the baseline sample values (e.g., the sample values of the first portion of the input signals). The condition detection engine 114 can determine the delay as the amount of time between the activation of the thermal stimulator 106 and when the sample values within the second portion of the input signals return to within between about 1% and about 75%, between about 1% and about 50%, between about 1% and about 40%, between about 1% and about 30%, between about 1% and about 25%, between about 2% and about 25%, between about 5% and about 25%, or between about 10% and about 25% of the baseline sample values (e.g., the sample values of the first portion of the input signals). In some implementations the condition detection engine 114 can determine that an autonomic dysfunction exists if the reset time for returning to the baseline (or within a range of the baseline) is above a predetermined threshold. For example, the thermal unit 102 can include a data structure that includes reset times. The reset times can be indexed to patient parameters within the data structure. The patient parameters can include, for example, the subject's age, weight, sex, health condition, or combination thereof. The condition detection engine 114 can look up a reset from the data structure based on the subject's patient parameters. For example, the condition detection engine 114 can select a threshold based on the subject's age and sex. In some implementations the condition detection engine 114 can determine that the subject does not have an autonomic dysfunction if the reset time is less than the reset time selected from the data structure, and the condition detection engine 114 can determine that the subject does have an autonomic dysfunction if the reset time is greater than the reset time selected from the data structure.

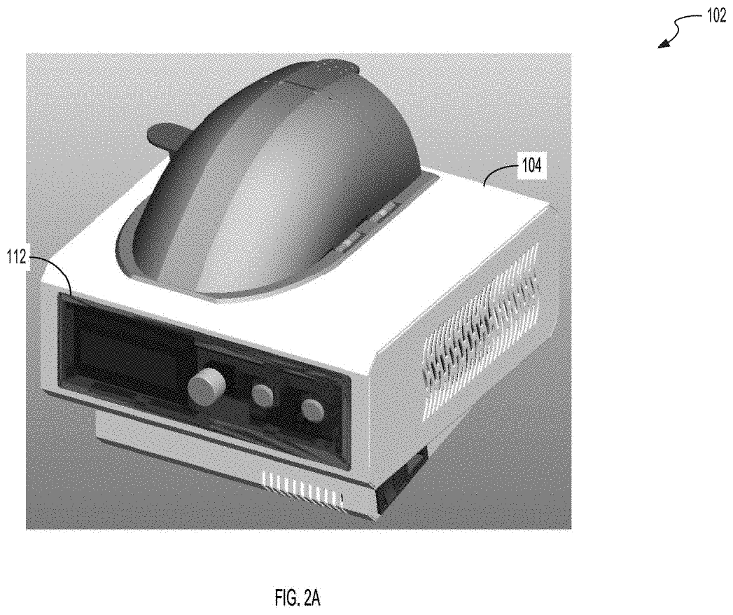

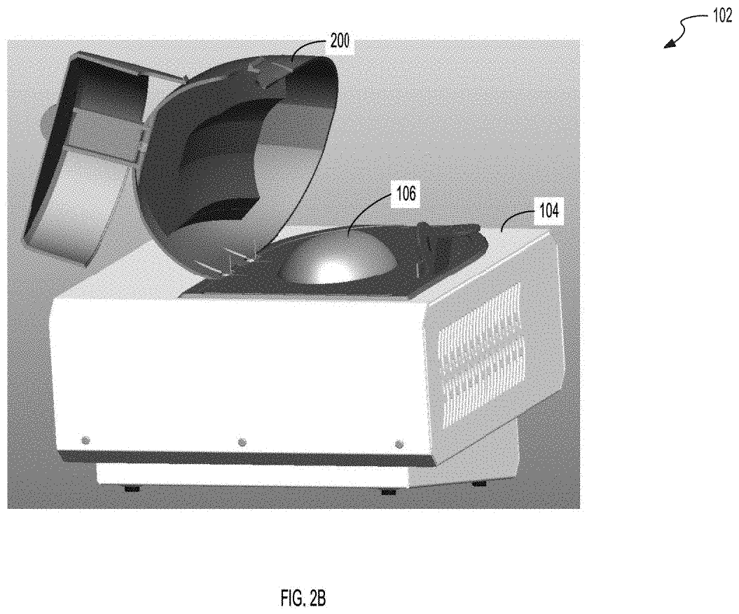

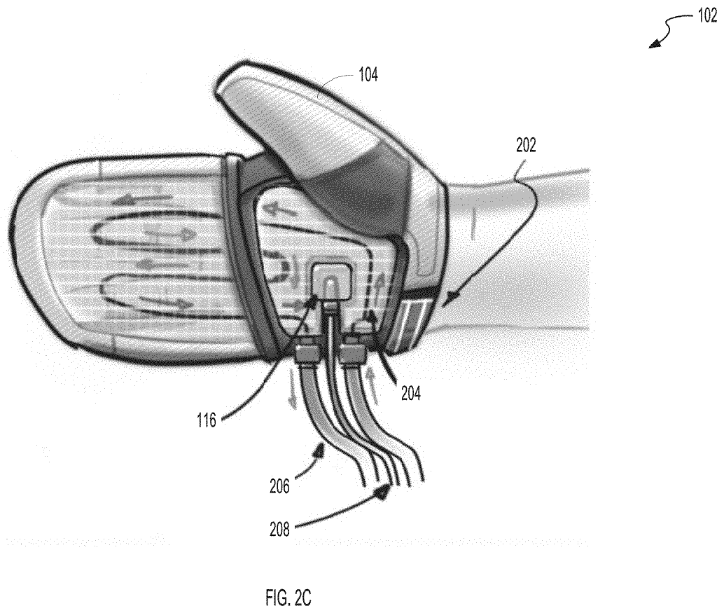

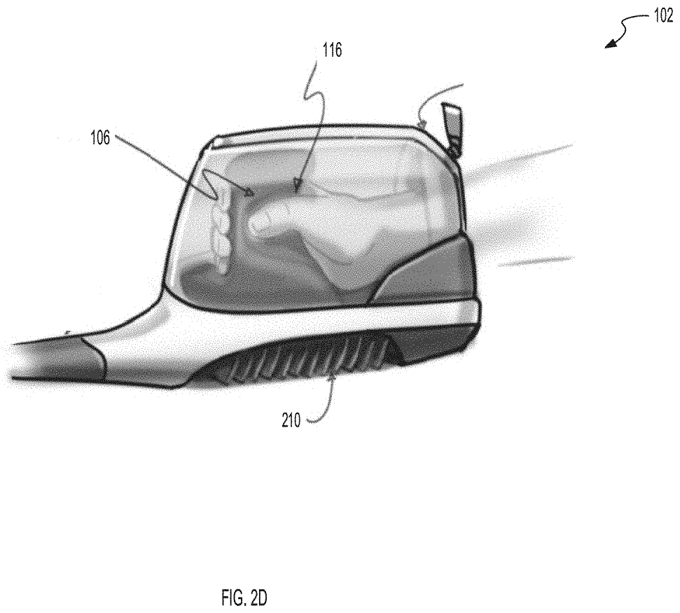

FIGS. 2A and 2B illustrate an example thermal unit 102. The thermal unit 102 can include an enclosure 104 that can house the condition detection engines 114, sensors 116, and controllers 118. In some implementations, the exterior of the thermal unit 102 can include an interface 112. The interface 112 can include interface elements, such as, but not limited to, screens, buttons, dials, and knobs. A user of the thermal unit 102 can set, via the interface 112, a temperature of the thermal stimulus. The user can set, via the interface 112, a length of time that the subject 108 is exposed to the thermal stimulation.

The enclosure 104 can include a cover 200 that a health care professional or the subject can open to expose the thermal stimulator 106. The user can at least partially close the cover 200 around the, for example, hand of the subject 108. While the hand is within the partially closed cover 200, the subject's hand can be placed on the thermal stimulator 106. The thermal stimulator 106 can be contoured. For example, the thermal stimulator 106 can be dome-shaped to contour to the shape of the subject's hand.

FIG. 2C illustrates an example configuration of the thermal unit 102. As illustrated in FIG. 2C, the enclosure 104 is configured as a glove. The glove can be worn on the hand of the subject 108. The glove can include a closure 202 that enables the glove to be sealed around the wrist of the subject 108. The glove can include tubing 204 that can run through the interior of the glove. The tubing 204 can be filled with a liquid. The liquid can be an antifreeze, non-freezing gel, water, or other fluid. The liquid can be supplied to the glove via external tubing 206. The thermal stimulator 106 can cool the liquid that flows through the external tubing 206 and into the tubing 204 to expose the subject's hand to the thermal stimulus. The glove can include a sensor 116, which can be a temperature sensor. The temperature sensor can measure the temperature of the thermal stimulus at the subject's hand. The sensor 116 can be coupled with the controller 118 via a wire 208.

FIG. 2D illustrates an example configuration of the thermal unit 102. The enclosure 104 can be or can include a box-shaped enclosure. The enclosure 104 can include the thermal stimulator 106. The thermal stimulator 106 can be contoured as a hand grip. The subject can grip the hand-gripped contoured thermal stimulator 106. The thermal stimulator 106 can include a sensor 116 that measures the temperature of the thermal stimulus as the thermal stimulator 106 is cooled while the subject 108 grasps the thermal stimulator 106.

While FIGS. 2A-2D illustrate example configurations of the thermal unit 102 that provide the thermal stimulation to the subject's hand, the thermal unit 102 can be configured to provide the thermal stimulation to other portions of the subject's body. FIGS. 2A-2D are provided for illustrative purposes only and do not limit where the thermal stimulus can be applied. The thermal unit 102 can be configured to provide the thermal stimulation to the subject's foot, leg, groin, abdomen, arm, underarm, wrist, back, neck, or head. For example, the thermal unit can be configured to wrap around the subject's neck and provide the thermal stimulation to the back of the subject's neck.

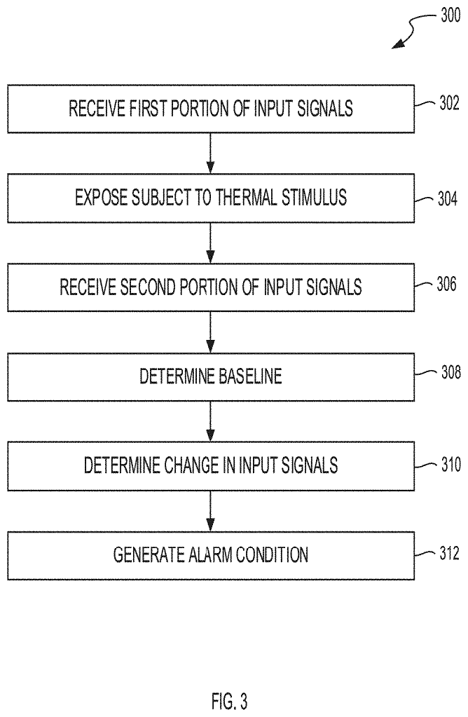

FIG. 3 illustrates a block diagram of an example method 300 to diagnose autonomic dysfunction. The method 300 can include receiving a first portion of one or more input signals (BLOCK 302). The method 300 can include exposing a subject to a thermal stimulus (BLOCK 304). The method 300 can include receiving a second portion of one or more input signals (BLOCK 306). The method 300 can include determining a baseline in the first portion of one or more input signals (BLOCK 308). The method 300 can include determining a change in the second portion of the one or more input signals (BLOCK 310). The method 300 can include generating an alarm condition (BLOCK 312).

The method 300 can include receiving a first portion of one or more input signals (BLOCK 302). Also referring to FIG. 1, among others, the condition detection engine 114 can receive, identify, or retrieve an input signal. The input signal can be generated by the monitors 110. The input signals can include vital signs of the subject 108. For example, the input signals can include blood pressure measurements, heart rate measurements, electrocardiogram measurements, or other biometric measurements. The blood pressure measurements can include beat-to-beat blood pressure measurements. The vital sign can be a derived vital sign. For example, the derived vital sign can be calculated from one or more of blood pressure measurements, heart rate measurements, electrocardiogram measurements, or other biometric measurements. For example, the derived vital sign can be R to R heart rate interval, a heart rate variability, or a root mean squared value of the heart rate. In some implementations, the heart rate, R to R heart rate interval, heart rate variability, and root mean squared value can be derived from an input signal that can include an electrocardiogram signal.

The first portion of the input signals can include a baseline reading. For example, the subject 108 can place the subject's hand within the enclosure 104 and in contact with the thermal stimulator 106. During the initial phase, when the first portion of the input signals is recorded, the thermal stimulator 106 can be inactive (e.g., the subject 108 is not exposed to the thermal stimulus). In some implementations, the condition detection engine 114 can determine a baseline for each of the input signals that includes a different vital sign.

In some implementations, the first portion of the input signal can be an input signal recorded during a recording session before the session where the subject is exposed to the thermal stimulus. For example, a person at risk of a concussion (e.g., a football player) can have a baseline recording prepared before a possible injury occurs (e.g., at the start of the search for the football player). During the baseline recording, the subject's vital signs can be recorded and stored. In some implementations, the subject 108 can be exposed to the thermal stimulus during the baseline recording such that the condition detection engine 114 can compare the magnitude and timing of the subject's physiological response prior to trauma (or a disease progression) to the magnitude and timing of the subject's physiological response following the trauma (or disease progression).

The method 300 can include exposing the subject 108 to a thermal stimulus (BLOCK 304). As described above in relation to FIG. 2, among others, the subject 108 can insert their hand into the thermal unit 102. The subject 108 can place their hand in contact with the thermal stimulator 106 and a health care professional can activate the thermal unit 102. The controller 118 can start a timer for the baseline recording captured by the monitors 110 in the first portion of the input signals. After a predetermined amount of time (e.g., 2 minutes), the controller 118 can activate the thermal stimulator 106, cool the thermal stimulator 106, and expose the subject to the thermal stimulus. For example, the thermal stimulator 106 can include a heat pump, which when activated, reduces the surface temperature of the thermal stimulator 106 to between about −10° C. and about 10° C.

The method 300 can include receiving a second portion of one or more input signals (BLOCK 306). The condition detection engine 114 can receive, identify, or retrieve the second portion of the input signals. The second portion of the input signal can be a continuation of the first portion of the input signals. In some implementations, the thermal unit 102 can receive a first set of input signals from the monitors 110 prior to the controller 118 activating the thermal stimulator 106 and a second set of input signals from the monitors 110 after the controller 118 activates the thermal stimulator 106. The second portion of the input signals can include vital signs of the subject 108. For example, the input signals can include blood pressure measurements, heart rate measurements, electrocardiogram measurements, or other biometric measurements. In some implementations, the second portion of the input signals can include the subject's physiological response to the thermal stimulus. For example, the monitors 110 can capture in the input signals an increase in the subject's heart rate and blood pressure in response to the thermal stimulator 106 exposing the subject to the thermal stimulus. In some implementations, the condition detection engine 114 can divided the second portion of the input signals into a third portion. The third portion of the input signals can include a portion of the input signals after the thermal stimulator 106 is deactivated. For example, the controller 118 can output a signal indicating a status of the thermal stimulator 106. The output signal can include zero values initially to indicate the thermal stimulator 106 is off, non-zero values when the controller 118 activates the thermal stimulator 106, and zero values when the controller 118 deactivates the thermal stimulator 106. The first portion of the input signals can correspond to when the thermal stimulator 106 was initial deactivated, the second portion of the input signals can correspond to when the thermal stimulator 106 was active, and the third portion of the input signals can correspond to when the thermal stimulator 106 was deactivated after the active portion. In some implementations, the second portion can be or can include a portion of the input signals after the thermal stimulator 106 is deactivated.

The method 300 can include determining a baseline (BLOCK 308). The condition detection engine 114 can determine a baseline for the first portion of the input signals. The condition detection engine 114 can determine the baseline as, for example, an average of each of the input signals during the first portion of the input signals before exposure of the thermal stimulus to the subject.

The method 300 can include determining a change in the second portion of the one or more input signals (BLOCK 310). The second portion of the one or more input signals can be the portion of the input signals that captures the subject's response to the thermal stimulus. For example, the condition detection engine 114 can mark the start of the second portion of the input signals as the time that the controller 118 activated the thermal stimulator 106. In some implementations, the condition detection engine 114 can determine a change between the second portion of the one or more input signals and an earlier recorded baseline recording. The condition detection engine 114 can calculate a change from the baseline determined during the first portion of the input signals. In some implementations, the condition detection engine 114 can determine an amount or magnitude of a change from the baseline that is present in the second portion of the input signals. In some implementations, the condition detection engine 114 can determine a change based on a response time or delay that the condition detection engine 114 detects during the second portion of the input signals. For example, the condition detection engine 114 can detect a time between the activation of the thermal stimulator 106 and a change in the vital signs measured by the monitors 110. For example, a delay or time between the controller's activation of the thermal stimulator 106 and an increase in the subject's average heart rate. In some implementations, the condition detection engine 114 can determine a reset time or an amount of time between the activation of the thermal stimulator 106 and the time it takes for the vital signs to return to the level determined during the baseline.

The method 300 can include generating an alarm condition (BLOCK 312). The thermal unit 102 can generate an alarm condition based on the change from baseline determined at BLOCK 310. The alarm condition can be included in a report provided to the user of the thermal unit 102 (e.g., a medical professional). The alarm condition can include an indication of the classification or type of the autonomic dysfunction. In some implementations, the alarm condition can be displayed to the user through a user interface. For example, the thermal unit 102 can include a screen or light emitting diode (LEDs) that can indicate to the user when the thermal unit 102 determines the subject 108 has an autonomic dysfunction.

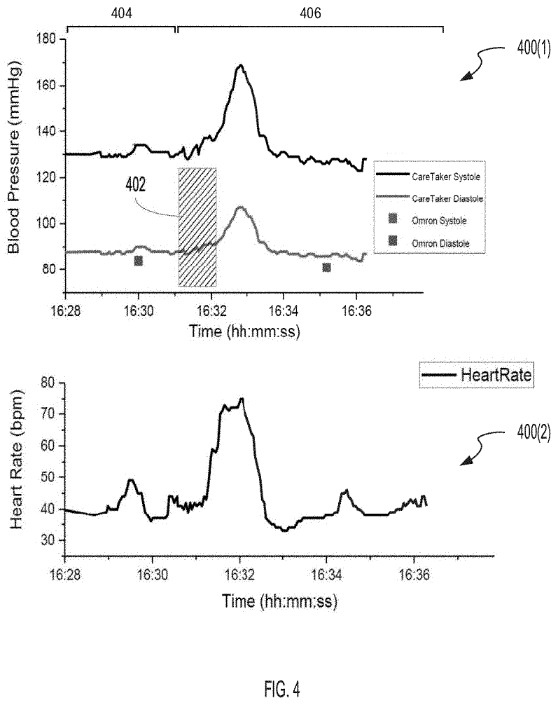

FIG. 4 illustrates plots of example input signals. FIG. 4 illustrates a plot 400(1) of the input signals of the subject's blood pressure and a plot 400(2) of the input signals of the subject's heart rate. The plot 400(1) of the subject's blood pressure includes the subject's systolic and diastolic blood pressure. As illustrated in the plots, the subject is exposed to the thermal stimulus during time period 402. The condition detection engine 114 can divide the input signal into a first portion 404 and a second portion 406. The first portion 404 can end, and the second portion 406 can begin at, the start of the time period 402 when the subject is exposed to the thermal stimulation. In some implementations, the thermal unit 102 can save the first portion 404 and the second portion 406 as separate files. For example, as the controller 118 receives the input signal from the monitors 110, the controller 118 can write the samples of the input signals to a first file prior to the activation of the thermal stimulator 106 (which can be or can include the first portion 404), and the controller 118 can write the samples of the input signals to a second file after the activation of the thermal stimulator 106 (which can be or can include the second portion 406). In some implementations, as described above, the thermal unit 102 can write the first portion 404 and the second portion 406 to a single file.

While operations are depicted in the drawings in a particular order, such operations are not required to be performed in the particular order shown or in sequential order, and all illustrated operations are not required to be performed. Actions described herein can be performed in a different order.

The separation of various system components does not require separation in all implementations, and the described program components can be included in a single hardware or software product.

Having now described some illustrative implementations, it is apparent that the foregoing is illustrative and not limiting, having been presented by way of example. In particular, although many of the examples presented herein involve specific combinations of method acts or system elements, those acts, and those elements may be combined in other ways to accomplish the same objectives. Acts, elements, and features discussed in connection with one implementation are not intended to be excluded from a similar role in other implementations or implementations.

The phraseology and terminology used herein is for the purpose of description and should not be regarded as limiting. The use of “including,” “comprising,” “having,” “containing,” “involving,” “characterized by,” “characterized in that,” and variations thereof herein is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

As used herein, the terms “about” and “substantially” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

Any references to implementations or elements or acts of the systems and methods herein referred to in the singular may also embrace implementations including a plurality of these elements, and any references in plural to any implementation or element or act herein may also embrace implementations including only a single element. References in the singular or plural form are not intended to limit the presently disclosed systems or methods, their components, acts, or elements to single or plural configurations. References to any act or element being based on any information, act, or element may include implementations where the act or element is based at least in part on any information, act, or element.

Any implementation disclosed herein may be combined with any other implementation or embodiment, and references to “an implementation,” “some implementations,” “one implementation,” or the like are not necessarily mutually exclusive and are intended to indicate that a particular feature, structure, or characteristic described in connection with the implementation may be included in at least one implementation or embodiment. Such terms as used herein are not necessarily all referring to the same implementation. Any implementation may be combined with any other implementation, inclusively or exclusively, in any manner consistent with the aspects and implementations disclosed herein.

The indefinite articles “a” and “an,” as used herein in the specification and in the claims, unless clearly indicated to the contrary, should be understood to mean “at least one.”

References to “or” may be construed as inclusive so that any terms described using “or” may indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items.

Where technical features in the drawings, detailed description, or any claim are followed by reference signs, the reference signs have been included to increase the intelligibility of the drawings, detailed description, and claims. Accordingly, neither the reference signs nor their absence have any limiting effect on the scope of any claim elements.

The systems and methods described herein may be embodied in other specific forms without departing from the characteristics thereof. The foregoing implementations are illustrative rather than limiting of the described systems and methods. Scope of the systems and methods described herein is thus indicated by the appended claims, rather than the foregoing description, and changes that come within the meaning and range of equivalency of the claims are embraced therein.

What is claimed:

1. A system, comprising:
a thermal unit to maintain a predetermined temperature and expose a subject to a thermal stimulus;
an interface to receive one or more input signals indicating one or more vital signs of the subject in response to exposure to the thermal stimulus, wherein the vital signs comprise at least one of a blood pressure of the subject or a heart rate of the subject; and
a condition detection engine to:
determine a baseline of the one or more vital signs over a first portion of the one or more input signals prior to the exposure to the thermal stimulus;
detect, at a second portion of the one or more input signals after a start of the exposure to the thermal stimulus, a change from the baseline of the one or more vital signs over the first portion of the one or more input signals; and
generate an alarm condition indicating an autonomic dysfunction based on the change from the baseline of the one or more vital signs over the first portion of the one or more input signals.

2. The system of claim 1, wherein the autonomic dysfunction is a concussion.

3. The system of claim 1, wherein the baseline of the one or more vital signs comprises a first baseline of the heart rate of the subject and a second baseline of the blood pressure of the subject.

4. The system of claim 1, wherein the condition detection engine is further configured to:
determine a first baseline of the heart rate over the first portion of the one or more input signals;
determine a second baseline of the blood pressure over the first portion of the one or more input signals; and
generate the alarm condition indicating the autonomic dysfunction based on a change of the first baseline during the second portion of the one or more input signals and a change of the second baseline during the second portion of the one or more input signals.

5. The system of claim 1, wherein the condition detection engine is further configured to:
determine a baseline of the one or more vital signs over a portion of one or more control input signals received that are generated prior to the interface receiving the one or more input signals indicating one or more vital signs of the subject in response to exposure to the thermal stimulus; and
generate the alarm condition indicating the autonomic dysfunction based on a change from the baseline of the one or more vital signs over a portion of one or more control input signals received that are generated prior to the interface to receive one or more input signals indicating one or more vital signs of the subject in response to exposure to the thermal stimulus.

6. The system of claim 1, wherein the condition detection engine is further configured to determine a derived vital sign based on the one or more vital signs of the subject.

7. The system of claim 6, wherein the derived vital sign comprises an R to R heart rate interval, a heart rate variability, or a root mean squared value of the heart rate.

8. The system of claim 1, wherein the thermal stimulus is between −10° C. and 10° C.

9. The system of claim 1, wherein the thermal unit comprises a thermoelectric thermal stimulator to maintain the thermal stimulus.

10. The system of claim 1, wherein the thermal unit comprises a thermal stimulator conformed to receive a hand of the subject and expose the hand of the subject to the thermal stimulus.

11. A method, comprising:
receiving a first portion of one or more input signals indicating one or more vital signs of a subject, wherein the vital signs comprise at least one of a blood pressure of the subject or a heart rate of the subject;
exposing the subject to a thermal stimulus;
receiving a second portion of the one or more input signals indicating the one or more vital signs of the subject, wherein the second portion of the one or more input signals comprises a response to the thermal stimulus;
determining a baseline of the first portion of one or more input signals indicating the one or more vital signs of the subject;
determining a change from the baseline of the one or more vital signs over the first portion of the one or more input signals in the second portion of the one or more input signals; and
generating an alarm condition indicating an autonomic dysfunction based on the change from the baseline of the one or more vital signs over the first portion of the one or more input signals in the second portion of the one or more input signals.

12. The method of claim 11, wherein the autonomic dysfunction is a concussion.

13. The method of claim 11, wherein the baseline of the one or more vital signs comprises a first baseline of the heart rate of the subject and a second baseline of the blood pressure of the subject.

14. The method of claim 11, further comprising:
determining a first baseline of the heart rate over the first portion of the one or more input signals;
determining a second baseline of the blood pressure over the first portion of the one or more input signals; and
generating the alarm condition indicating the autonomic dysfunction based on a change of the first baseline during the second portion of the one or more input signals and a change of the second baseline during the second portion of the one or more input signals.

15. The method of claim 11, further comprising:
determining a baseline of the one or more vital signs over a portion of one or more control input signals received that are generated prior to the interface receiving the one or more input signals indicating one or more vital signs of the subject in response to exposure to the thermal stimulus; and
generating the alarm condition indicating the autonomic dysfunction based on a change from the baseline of the one or more vital signs over a portion of one or more control input signals received that are generated prior to the interface to receive one or more input signals indicating one or more vital signs of the subject in response to exposure to the thermal stimulus.

16. The method of claim 11, determining a derived vital sign based on the one or more vital signs of the subject.

17. The method of claim 16, wherein the derived vital sign comprises an R to R heart rate interval, a heart rate variability, or a root mean squared value of the heart rate.

18. The method of claim 11, wherein the thermal stimulus is between −10° C. and 10° C.

19. The method of claim 11, further comprising exposing a hand of the subject to the thermal stimulus.

20. The method of claim 11, further comprising exposing the subject to the thermal stimulus for between 1 minute and 5 minutes.
